(19) United States
(12) Patent Application Publication
(10) Pub. No.: US 2025/0144075 A1
(43) Pub. Date: May 8, 2025

(54) Melanin for treating lipofuscin-associated diseases

(71) Applicant: Katairo GmbH, Kusterdingen (DE)

(72) Inventor: Ulrich SCHRAERMEYER, Hechingen (DE)

(73) Assignee: Katairo GmbH, Kusterdingen (DE)

(21) Appl. No.: 18/837,398

(22) PCT Filed: Feb. 9, 2023
(86) PCT No.: PCT/EP2023/053262

§ 371 Date: Aug. 9, 2024

Related U.S. Application Data
(63) Continuation-in-part of application No. PCT/EP2022/053137, filed on Feb. 9, 2022.

(51) Int. Cl. A61K 31/407; A61K 31/34; A61K 31/4375

(52) U.S. Cl. CPC A61K 31/407; A61K 31/34; A61K 31/4375

(57) ABSTRACT

The present invention relates to a composition for use in the treatment and/or prophylaxis of a lipofuscin-associated disease, wherein the composition comprises melanin and/or induces melanogenesis.

### FIELD OF THE DISCLOSURE

The present invention concerns the field of lipofuscin-associated diseases. It provides compositions for the treatment and/or prophylaxis of lipofuscin-associated diseases. These compositions comprise melanin and/or induce melanogenesis. The compositions also comprise combinations of melanin and/or melanin inducing agents with superoxide- and/or NO generating compounds.

### BACKGROUND OF THE INVENTION

Lipofuscin is a general term to describe fine yellow-brown pigment granules composed of lipid-containing residues of lysosomal digestion. It is considered to be one of the aging or “wear-and-tear” pigments, found in the liver, kidney, heart muscle, retina, adrenals, nerve cells, and ganglion cells. It is specifically arranged around a nucleus, and is a type of lipochrome.

In its broadest sense, the accumulation of critical amounts of lipofuscin is pathologic in any tissue, but especially so in the tissues of the CNS where the loss of cell function through lipofuscin is particularly apparent.

The composition of lipofuscin is complex and is still under investigation. It appears to be the product of the oxidation of unsaturated fatty acids and may be symptomatic of membrane damage, or damage to mitochondria and lysosomes. Lipofuscin is known to contain sugars and metals, including mercury, aluminium, iron, copper and zinc. Lipofuscin is also accepted as comprising oxidized proteins (30-70%) as well as lipids (20-50%).

In the eye, one important and well characterized component of lipofuscin is the fluorophore N-retinylidene-N-retinylethanolamine (A2E), a byproduct of the visual cycle. A2E is often used in research for mimicking lipofuscin. It can be detected histologically by its autofluorescence properties.

The accumulation of lipofuscin-like material may be the result of an imbalance between formation and disposal mechanisms.

Lipofuscinoses and lipofuscinopathies are diseases characterized by high levels of lipofuscin deposits as a result of aging, or metabolic defects. Lipofuscin-associated degenerative diseases of the eye have in common that lipofuscin is accumulated in the cells of the RPE. Such diseases include age-related macular degeneration, Stargardt's disease, Best's disease and subpopulations of Retinitis pigmentosa (RP).

Retinal lipofuscinopathy is a general term describing the accumulation of lipofuscin in the RPE which may result in degeneration of the retina and vision loss.

AMD is a medical condition which may result in blurred or no vision in the center of the visual field. The pathogenesis of age-related macular degeneration is not well known, although some theories have been put forward, including oxidative stress, mitochondrial dysfunction, and inflammatory processes. The imbalance between the production of damaged cellular components and degradation leads to the accumulation of harmful products, for example, intracellular lipofuscin and extracellular drusen. Incipient atrophy is demarcated by areas of retinal pigment epithelium (RPE) thinning or depigmentation that precede geographic atrophy in the early stages of AMD. In advanced stages of AMD, atrophy of the RPE (geographic atrophy) and/or development of new blood vessels (neovascularization) result in the death of photoreceptors and central vision loss. In the dry (nonexudative) form, cellular debris called drusen accumulates between the retina and the choroid, causing atrophy and scarring to the retina. In the wet (exudative) form, which is more severe, blood vessels grow up from the choroid (neovascularization) behind the retina which can leak exudate and fluid and also cause haemorrhaging.

Morbus Stargardt or Stargardt's disease is the most common inherited single-gene retinal disease. It usually has an autosomal recessive inheritance caused by mutations in the ABCA4 gene. Rarely, it has an autosomal dominant inheritance due to defects with ELOVL4 or PROM1 genes. It is characterized by macular degeneration that begins in childhood, adolescence or adulthood, resulting in progressive loss of vision.

Vitelliform macular dystrophy or Best's disease, is an irregular autosomal dominant eye disorder and a retinal lipofuscinosis which can cause progressive vision loss. This disorder affects the retina, specifically cells in a small area near the center of the retina called the macula. Mutations in either the VMD2 or RDS gene can cause the adult-onset form of vitelliform macular dystrophy; however less than a quarter of cases result from mutations in these two genes. In most cases, the cause of the adult-onset form is unknown.

Retinitis pigmentosa (RP) is a genetic disorder of the eyes that causes loss of vision. It is generally inherited from a person's parents. Mutations in one of more than 50 genes are involved. The underlying mechanism involves the progressive loss of rod photoreceptor cells in the back of the eye. This is generally followed by loss of cone photoreceptor cells. Abnormal levels of lipofuscin accumulation are observed in more than one half of RP patients.

Lipofuscin-associated diseases are also found in other tissues. In the peripheral nervous, system, abnormal accumulation of lipofuscin or lipofuscinosis, respectively, are associated with a family of neurodegenerative disorders-neuronal ceroid lipofuscinoses, the most common of these is Batten disease. Also, pathological accumulation of lipofuscin is implicated in Alzheimer's disease, Parkinson's disease, amyotrophic lateral sclerosis, certain lysosomal diseases, acromegaly, denervation atrophy, lipid myopathy, chronic obstructive pulmonary disease, and centronuclear myopathy. Accumulation of lipofuscin in the colon is the cause of the condition melanosis coli.

It has been found that the lipofuscin component can be degraded by a variety of radical generating compounds or by induction of chemical reactions during which radicals are formed in monkeys and Abca4^{-/-} mice. Among them are the superoxide generators Soraprazan (see e.g. Kim, H. J., et al., Bisretinoids of the Retina: Photo-Oxidation, Iron-Catalyzed Oxidation, and Disease Consequences. Antioxidants, 2021. 10(9): p. 1382 and Julien, S. and U. Schraermeyer, Lipofuscin can be eliminated from the retinal pigment epithelium of monkeys. Neurobiology of Aging, 2012. 33: p. 2390-2397) and Riboflavin (see e.g. Schraermeyer, U., et al., Degradation of lipofuscin in Stargardt mice can be enhanced by the superoxide generator riboflavin—a hypothesis for melanolipofuscin formation. Investigative Ophthalmology & Visual Science, 2019. 60(9)), light (see e.g. Ueda, K., et al., Photodegradation of retinal bisretinoids in mouse models and implications for macular degeneration. Proc Natl Acad Sci USA, 2016(113): p. 6904-6909), and peroxidase (see e.g. Wu, Y., et al., Enzymatic degradation of A2E, a retinal pigment epithelial lipofuscin bisretinoid. J Am Chem Soc, 2011. 133(4): p. 849-57). Additionally, the radical generator Visodyne (verteporphin) which is originally used to destroy pathological blood vessels in wet AMD removed the lipofuscin component from melanolipofuscin granules in Abca4/mice after intravitreal injection efficiently (see e.g. WO 2015/121441 A1).

Melanin has unusual redox properties. It can both generate and absorb radicals. Melanin can change any energy into electric energy (electron flow). Even gamma radiation can be converted by melanin into chemical energy for growth by radiotrophic fungi detected for example in Tschernobyl. Melanin is an O2 donator and serves as a naturally occurring biological source of electrons to power biochemical reactions. Electrons degrade retinoids and bisretinoids by electrolysis. Melanosomes, the organelles in which the melanin is formed, are specialized lysosomes and contain many lysosomal enzymes. The natural function of lysosomes is the degradation of molecules. Melanosomes of the RPE in mammals are only formed before birth and lack any turnover during life.

Aged oxidized melanin may accumulate in the retinal pigment epithelium and certain diseases, including lipofuscin-associated diseases, might be associated therewith.

So far there is no therapy available against lipofuscin-associated diseases which did prove therapeutically meaningful in practice. In particular, most of the currently used or proposed pharmacological substances are characterized by severe side effects, such as cytotoxicity, which render them unsuitable for use in a human patient.

Against this background it was an object underlying the present invention to provide a composition for use in the treatment and/or prophylaxis of a lipofuscin-associated disease by means of which the problems of the current therapies or compounds are avoided or at least reduced.

The present invention satisfies these and other needs.

### SUMMARY OF THE INVENTION

It has surprisingly been found that melanin and compositions comprising melanin allow for an effective treatment and/or prophylaxis of a lipofuscin-associated disease due to melanin being able to remove excessive lipofuscin. It has further been found that melanin in combination with a superoxide- and/or a NO-generator provide a surprising synergistic effect of removal.

The present invention provides a composition for use in the treatment of a lipofuscin-associated disease, wherein the composition comprises melanin and/or induces melanogenesis.

### BRIEF DESCRIPTION OF THE FIGURES

FIG. 1 Lipid peroxides (arrowheads) which are considered to be the main cause of lipofuscin formation are present within the melanosomes of the RPE in a rat. Lipid peroxides are taken up with the shedded tips from the photoreceptor outer segments and then transported to the melanosomes.

FIG. 2 One week after subretinal injection of melanosomes into Wistar rat eyes the pigment granules can be seen in choroidal melanocytes (arrow) and in the RPE cell layer which both normally are amelanotic in albinos. Surprisingly at the Ora serrata (indicated by a black arrowhead) the layer of the ciliary body pigment epithelium (CBE) also contains melanosomes. The non-pigmented layer of the ciliary epithelium (white arrowhead) has not taken up melanin granules. In rats close to the Ora serrata the retina frequently does not contain photoreceptors and Bruch's membrane (BM) is somewhat separated from the pigment epithelium.

FIG. 3 Electron micrographs with the same magnification of the isolated melanosomes before injection (left) and one week after injection within the RPE cells (middle) and within choroidal melanocytes (right) are shown. The size of the melanosomes is clearly reduced in the melanocytes (right) which indicates degradation.

FIG. 4 In choroidal melanocytes the melanosomes were degraded much quicker than in the RPE. The choroidal melanocytes (arrows) degrade the melanosomes whereas the RPE cells did not. This is indicated by the reduction of the size of the melanosomes in melanocytes which was not seen in RPE cells.

FIG. 5 In a RPE cell of a Wistar rat the pigmentation was maintained during the observation period of 4 months. The photoreceptors and other retinal cells were well preserved which can be concluded by the structure of the outer segments (ROS). Most melanosomes appeared completely normal as in wild type animals.

FIG. 6 In RPE cells the pigmentation was maintained during the observation period of 4 months and the amount of pigmentation was locally different. In this example the pigmentation is even higher than in wild type rats. The photoreceptors and other retinal cells were well preserved which could be concluded by the structure of the outer segments (ROS).

FIG. 7 Melanosomes appeared completely normal as in wild type animals and were clearly surrounded by an own membrane (arrows) which indicates that they are fully functional which is shown here in a high power electron micrograph.

FIG. 8 The electron micrograph shows a RPE cell of a Wistar rat 5 weeks after injection of 0.5 μl soluble melanin which has been synthesized from DOPA. Melanin (arrow) is present in the RPE cells and did not show any adverse effects to retinal cells.

FIG. 9 (a) An unstained semi thin section is shown in a bright field image from an albino Abca4^{(-/-)} mouse 2 weeks after subretinal injection of tyrosinase vector. Melanin granules can be seen within the choroid (asterisks) and in the RPE (white arrows). (b) The same section as in (a) is shown under SW-AF in green AF. (c) The same section as in (a) shown under SW-AF in NIR-AF.

FIG. 10 (d) The merged image of both fluorescence modalities (green and NIR; see FIG. 9) is shown. In the RPE pigment granules are fluorescent only in SW (black arrowheads) or NIR or in both modalities (black arrow) or are lacking completely (white arrowheads). The granules in green represent lipofuscin, those in red represent melanin and those in yellow/orange are melanolipofuscin granules. Pigment granules that are fluorescent in both modalities appear yellow or orange. In the upper right part of the RPE cell layer (white arrowheads) the lipofuscin moieties have chemically reacted with the melanosomes. This reaction has been described as chemiexcitation of melanin (see Premi, S., et al., Photochemistry. Chemiexcitation of melanin derivatives induces DNA photoproducts long after UV exposure. Science, 2015. 347(6224): p. 842-7). During this reaction the melanin is oxidized which causes NIR-AF. The melanolipofuscin granules are then transferred to the choroidal cells, further degraded and finally removed via the blood vessels. The majority of the pigment granules in the choroid is fluorescent in both modalities although few melanin (red) or lipofuscin granules (green) can be identified. (e) The high amount of lipofuscin granules (white arrowheads) in the RPE of an age matched albino Abca4^{(-/-)} mouse without tyrosinase transfection under SW is shown.

FIG. 11A confocal scanning laser ophthalmoscope fundus AF image from an albino Abca4^{(-/-)} mouse 2 weeks after injection of tyrosinase vector shows spots of lipofuscin autofluorescence in the SW mode on the left (arrow). Some of these spots are also fluorescent in the NIR mode on the right (arrow). On the right side of the fundus the lipofuscin autofluorescence is reduced (asterisk) due to removal of this age pigment. Without tyrosinase injection NIR-AF is completely absent in albino Abca4^{(-/-)} mice (see Taubitz, T., et al., Ultrastructural alterations in the retinal pigment epithelium and photoreceptors of a Stargardt patient and three Stargardt mouse models: indication for the central role of RPE melanin in oxidative stress. PeerJ, 2018. 6: p. e5215). Ten out of 12 mice showed NIR-AF in the fundus.

FIG. 12 (A) A high power electron micrograph with 50000 fold magnification of a melanolipofuscin granule from a pigmented Abca4^{-/-} mouse is shown. The granule consists of a melanin (m) part and has an attachment of lipofuscin-like (I) material which is lesser electron-dense than the melanosome. In the upper part of this attachment, there are thin membranes forming lamellae of 8 nm distance (arrowheads). These thin membranes are regularly present in melanolipofuscin granules and the cytoplasm of RPE cells often associated with residual bodies. (B) TLMs can be found in Bruch's membrane (white arrow) and are present in endothelial cells of the choriocapillaris (black arrow) and the capillary lumen (not shown). (C) The residual body within the endothelial cell in shown in (B) is surrounded by thin membranes (arrowheads) at a high magnification of 140000 fold.

FIG. 13 (A) A high power electron micrograph with 50000 fold magnification of a melanolipofuscin granule from a pigmented Abca4^{-/-} mouse is shown. The granule consists of a melanin (m) part and has an attachment of lipofuscin-like (I) material which is lesser electron-dense than the melanosome. The content of the lipofuscin part here appears to be more homogeneously but in some areas, one still can recognize fine lamellar membranes (white arrowhead). The transition between melanin and lipofuscin is interwoven. A small fragment of the fine lamellar membrane is present in the cytoplasm (black arrowhead). The inset (D) shows the proportion between original photoreceptor disc membranes (upper part) and the condensed fine lamellar membranes under the same magnification (50000 fold). (B) shows a phagosome within an RPE cell of an albino Abca4^{-/-} mouse which underwent the reaction with tetramethylbenzidine as described earlier (see Kayatz, P., Heimann, K., Esser, P., Peters, S., & Schraermeyer, U. (1999). Ultrastructural localization of lipid peroxides as benzidine-reactive substances in the albino mouse eye. Graefes Arch Clin Exp Ophthalmol, 237(8), 685-690. https://doi.org/10.1007/s004170050297). Parts of the phagosomal membranes are now electron-dense (arrowhead). (C) The parts of the phagosome marked by the arrowhead in (B) are shown. It is obvious that the original photoreceptor disk membranes (white arrowhead) have fused and become condensed (black arrowhead).

FIG. 14 (A) and (B) Vacuole-like structures (asterisks) which are regularly filled with very heterogeneous types of material in albino Abca4/mice are shown. The majority of the material inside these vacuoles consists of fine lamellar membranes (white arrows; see also in C which appear electron-dense (white arrow) or electron opaque (black arrow) but flocculate residues are also present (asterisk in B). These vacuole-like structures can be separated towards the cytoplasm by a membrane (black arrowhead). But this membrane is incomplete and often absent (white arrowhead). (C) The fine lamellar nature (arrowhead) of these residual bodies can be seen in the high-power micrograph. (D): Quantification in electron microscopy revealed that vacuole-like structures are barely present in pigmented Abca4-(7 months old, n=3 eyes/group, p<0.0001).

FIG. 15 ARPE-19 cells were incubated with A2E (10 μM) for 9 h. Afterwards the solution was replaced by fresh cell culture medium overnight. Then the cells were substituted with cell culture medium containing 50 μg/ml melanin from Sepia officinalis (Sigma Aldrich, Taufkirchen, Germany) and 20 mg/ml of the nitric oxide donor isosorbid dinitrate (ISDN, Ratiopharm GmbH, Ulm, Germany) for 24h. As controls cells were incubated with ISDN (20 mg/ml) or melanin (50 μg/ml) alone for 24h.

### DETAILED DESCRIPTION OF THE INVENTION

The present invention provides compositions for use in the treatment and/or prophylaxis of lipofuscin-associated diseases. The individual aspects and suitable and preferred embodiments thereof will now be described in detail.

According to a first aspect the invention provides a composition for use in the treatment of a lipofuscin-associated disease. The composition comprises melanin and/or induces melanogenesis, i.e., is capable of inducing melanogenesis so that melanin is formed. The melanin interacts with lipofuscin, most preferably the melanin degrades lipofuscin and/or causes removal of lipofuscin. In a particular embodiment, the composition removes thin lamellar membranes (TLMs).

In a related aspect, the invention provides a method of treating a human being with a lipofuscin-associated disease, the method comprising administering to a subject in need thereof a composition comprising melanin and/or inducing melanogenesis. The melanin interacts with lipofuscin, most preferably the melanin degrades lipofuscin and/or causes removal of lipofuscin. In a particular embodiment, the composition removes thin lamellar membranes (TLMs).

In a preferred embodiment, the composition comprises melanin.

In an alternative embodiment the composition is capable of inducing melanogenesis. To this end the composition comprises at least one melanogenic component, i.e., a component that is capable of inducing melanogenesis. In this respect, it is preferred that the composition comprises a melanogenic enzyme, most preferably the composition comprises a tyrosinase. Alternatively, the composition comprises a gene that codes for a melanogenic enzyme, e.g. a gene that codes for a tyrosinase.

The term “lipofuscin-associated disease” as used herein refers to any kind of disease of an individual where, in comparison to a healthy reference individual, the lipofuscin level is altered. Such diseases include lipofuscinoses and lipofuscinopathies which are characterized by high levels of lipofuscin deposits, for instance as a result of aging, or metabolic defects. Also included are melanolipofuscin-associated diseases. Lipofuscin-associated diseases of the eye have in common that lipofuscin is accumulated in the cells of the regional pigment epithelium (RPE) and notably include retinal lipofuscinopathy, age-related macular degeneration (AMD), preferably in its dry form (dry AMD), Morbus Stargardt, vitelliform macular dystrophy (Best's disease) and Retinitis pigmentosa.

In a preferred embodiment the lipofuscin-associated disease is a disease of the eye or the CNS. It is particularly preferred that the lipofuscin-associated disease is a disease of the eye selected from age-related macular degeneration, Stargardt's disease, Best's disease or Retinitis pigmentosa, preferably the age-related macular degeneration is wet AMD or dry AMD, most preferably dry AMD.

A “living being” as used herein refers to any animal, including mammals and in particular humans.

In a particular embodiment, the compound or composition for use according to the invention is configured for an injection into a living being. As such, a dosage form is provided which allows an effective administration to the living being. “Injection” includes all kinds of parenteral administration, e.g. subcutaneous, intramuscular, intravenous, intraperitoneal, intraosseous, intracardiac, intraarticular, intracavernous, intravitreal and subretinal.

In a preferred embodiment, the injection is an intravenous, an intravitreal or a subretinal injection, preferably an intravitreal or a subretinal injection. This brings about the advantage of rapid availability and accumulation of the active ingredient at the site of action, namely at lipofuscin deposits or deposits of aged oxidized melanin and, in case of intravitreal or subretinal injection, a further reduction of systemic exposure to the composition. Therefore, the treatment and/or prophylaxis of a lipofuscin-associated disease is preferably by intravenous, intravitreal or subretinal injection of the composition, more preferably by intravitreal or subretinal injection.

In a preferred embodiment, the composition for use according to the invention comprises a pharmaceutically acceptable carrier. Pharmaceutically acceptable carriers are well known to the skilled person. They allow a proper formulation of the active agent and serve to improve the selectivity, effectiveness, and/or safety of drug administration. Pharmaceutically acceptable carriers include, without being limited thereto, solvents, fillers, binders, lubricants, stabilizers, surfactants, suspensions, thickeners, emulsifiers, preserving agents, liposomes, micelles, microspheres, nanoparticles, etc. suitable for the particular form of dosage. Except for cases, when the medium of conventional carriers is incompatible with the active ingredient, for example, upon occurrence of any undesirable biological effects or other adverse interactions with any other ingredient(s) of the pharmaceutical composition, the use of such compositions falls within the scope of this invention. Materials that can serve as pharmaceutically acceptable carriers include, but are not limited to, monosaccharides and oligosaccharides, as well as derivatives thereof; malt, gelatin; talc; excipients such as: cocoa butter and suppository waxes; oils, such as peanut oil, cottonseed oil, safflower oil, sesame oil, olive oil, corn oil and soybean oil; glycols such as propylene glycol; esters, such as ethyl oleate and ethyl laurate; agar; buffering agents, such as magnesium hydroxide and aluminium hydroxide; alginic acid; pyrogen-free water; isotonic saline, Ringer's solution; ethyl alcohol and phosphate buffer solutions. In addition, the composition may contain other non-toxic compatible lubricants, for example sodium lauryl sulfate and magnesium stearate, as well as coloring agents, parting liquids, film formers, sweeteners, flavoring additives and flavorants, preserving agents and antioxidants.

### EXAMPLES

### Example 1—Melanosomes are Involved in Detoxification of Lipid Peroxides

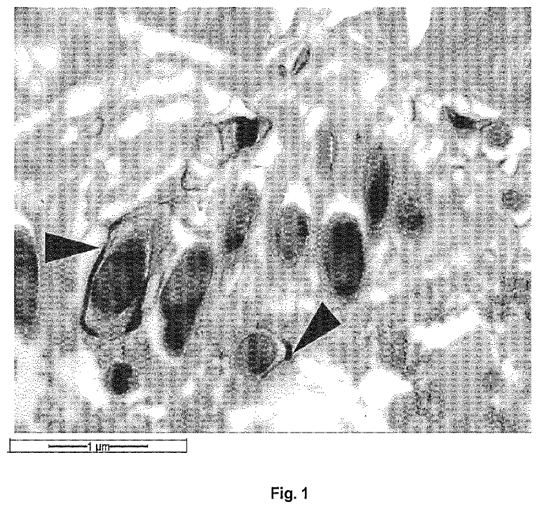

A method for detecting lipid peroxides (LP) at an ultrastructural level as benzidine-reactive substances (BRS) has been developed in the art. The commonly used techniques for detecting LP are biochemical methods that do not allow ultrastructural localization. In this experiment it is shown that the lipid peroxides which are considered to be the main cause of lipofuscin formation are present within the melanosomes of the RPE (FIG. 1) and phagosomes. These lipid peroxides are taken up with the shedded tips from the photoreceptor outer segments and then transported to the melanosomes.

The finding shows first evidence that melanosomes are involved in detoxification of lipid peroxides and possibly other undegradable metabolites of the retinol metabolism for example bisretionoids.

### Example 2—Melanosome Injection in Albino Rats

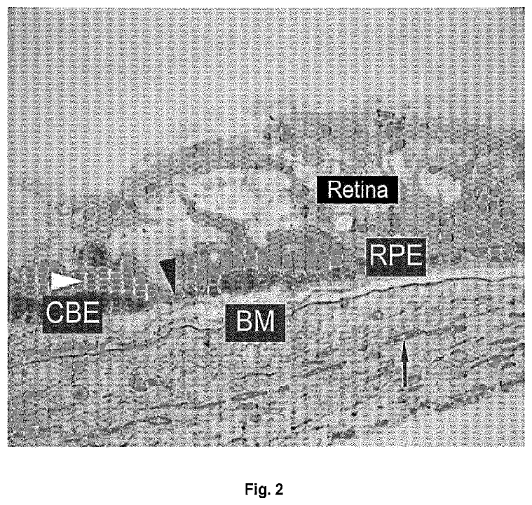

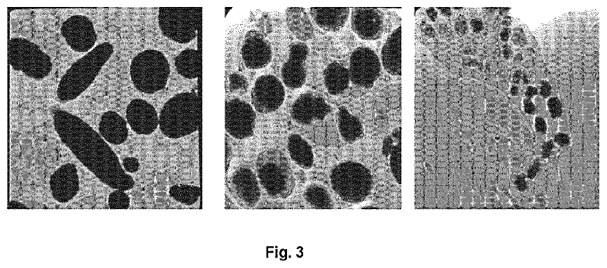

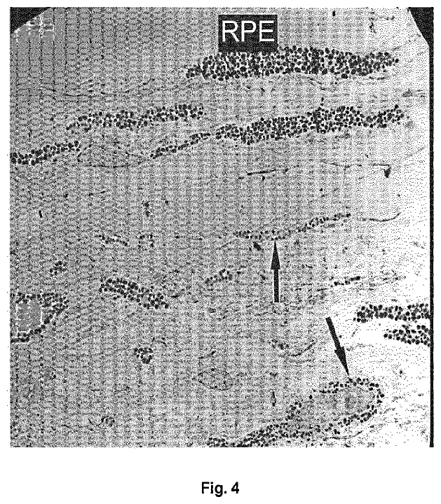

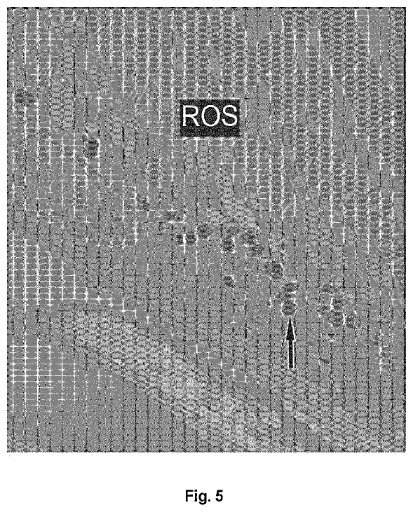

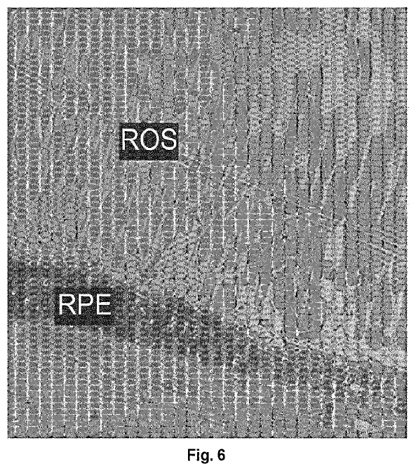

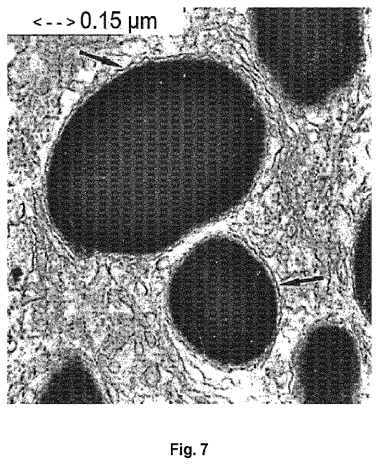

Isolated melanosomes from RPE cells of pigs were subretinally injected into albino Wistar rats. The eyes were enucleated and embedded for electron microscopy at different time points between 1 week and 4 months. One week after injection the melanosomes could be seen in choroidal melanocytes and in the RPE cell layer which both normally are amelanotic in albinos. Surprisingly at the Ora serrata the pigmented layer of the ciliary body pigment epithelium contains melanosomes (FIG. 2). In choroidal melanocytes the melanosomes were much more quickly degraded (FIGS. 3, 4) than in the RPE. FIG. 3 shows the isolated melanosomes before injection (left) and one week after injection within the RPE cells (middle) and within choroidal melanocytes (right). The choroidal melanocytes degrade the melanosomes whereas the RPE cells did not. This is indicated by the reduction of the size of the melanosomes in melanocytes which was not seen in RPE cells. All images of FIG. 3 have the same magnification. However in RPE cells the pigmentation was maintained during the observation period of 4 months and the amount of pigmentation was locally different (FIGS. 5, 6). The photoreceptors and other retinal cells were well preserved which could be concluded by the structure of the outer segments (FIGS. 5, 6). Most melanosomes appeared completely normal as in wild type animals and were clearly surrounded by an own membrane (FIG. 7) which indicates that they are fully functional. All in all the RPE, ciliary body epithelium and choroidal melanocytes appeared similar as in wild type animals after melanosome injection.

These results show that the increased susceptibility of albino animals to pathological lipofuscin accumulation is, at least in part, due to the lack of melanin. Based on this finding the intriguing possibility to employ melanin supplementation as a treatment strategy for lipofuscin-associated diseases was investigated further.

### Example 3—Subretinal Melanin Injection in Rats

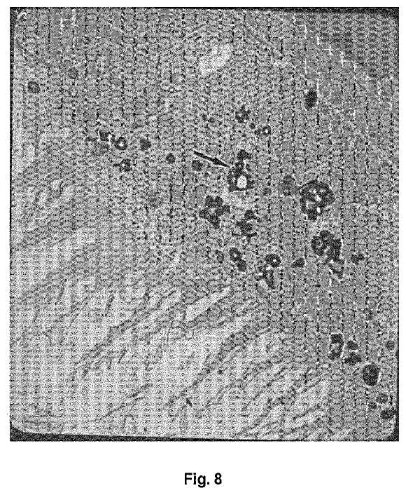

In a separate experiment 0.5 μl of soluble melanin which has been synthesized from DOPA (Sigma M-8631 Lot 10HO292) were also injected subretinally in Wistar rat. The eyes were enucleated after 5 weeks and embedded for electron microscopy. Melanin was present in the RPE cells (FIG. 8) and did not show any adverse effects to retinal cells.

From this experiment it can be concluded that injection of melanin is safe and hence administration of melanin might indeed represent a feasible treatment strategy.

### Example 4—Lipofuscin can be Removed from the RPE by Replacement with Melanin

In another set of experiments melanin synthesis was induced in albino Abca4^{(-/-)} mice by subretinal injection of a vector coding for tyrosinase. These mice lack the Abca4 flippase in their photoreceptors and therefore accumulate high amounts of lipofuscin in the RPE. Melanogenesis reduced the amount of lipofuscin in the RPE of these mice.

### Material and Methods

Generation of Ad-Tyr To generate the Ad-Tyr vector, the plasmid 123.B2 (kind gift from T. Woelfel, Mainz) was digested with EcoRI to release a 1,906-bp fragment containing the human tyrosinase cDNA. The fragment was blunt ended with Klenow and cloned into the EcoRV site of pCMVPac (+) under generation of pVI01. pCMVPac (+) is based on pCMVB (Invitrogen, Carlsbad, CA, USA) with two modifications: (1) the β-galactosidase coding sequence (NotI fragment) is replaced with a polylinker containing a unique EcoRV site and (2) upstream of the hCMV promoter and downstream of the SV40 polyA PacI sites are inserted which allow for release of the expression cassette. This plasmid serves as a tool to construct PacI-flanked expression cassettes driven by the hCMV promoter and containing an SV40 late 19s intron for strong ubiquitous expression of cDNAs. The PacI fragment from pVI01 containing the hCMV promoter, SV40 intron, human tyrosinase cDNA and SV40 polyA was isolated from pVI01 and cloned into PacI of pGS70 under generation of pVI02. Finally, the PacI fragment from pVI02 was cloned into PacI of pGS66 under generation of pVI03, an infectious adenovirus plasmid coding for the E1-deleted Ad vector Ad-Tyr. The virus backbone was released from pVI03 by digestion with SwaI and transfected into N52E6 cells. After appearance of a cytopathic effect, the virus vector was amplified on N52E6 cells and purified by double CsCl banding and desalting with PD-10 columns (Amersham Biosciences, Freiburg, Germany). The infectious and total particle titres were determined on A549 cells. Vector genome integrity was confirmed by restriction analysis of DNA prepared from purified virions. Expression of tyrosinase was confirmed in Western transfer experiments with cell lysates from A549 cells transduced with different amounts of Ad-Tyr.

Subretinal Injection with Ad-Tyr

Twenty-four 3-4 month old albino Abca4^{(+/-)} mice were anesthetized using isoflurane (Isoflurane CP®, CP-Pharma, Germany) inhalation (3.5% isoflurane and 25% oxygen). Pupils were fully dilated with mydriaticum drops (Pharmacy of the University of Tuebingen, Germany). One drop novesine (OmniVision, Puchheim, Germany) was applied as topical anesthetics before injection. The mice were positioned under a surgical microscope. Intravitreal injection was performed by inserting the tip of the syringe tangentially into eyes through the sclera into the sub retinal space without damaging the lens or posterior retina. One μl of Ad-Tyr (10⁸ iu/μl) was injected with a 34-gauge Hamilton syringe into the sub retinal space of the mice. After injection, antibiotic eye drops (Gentamicin-POS®, Ursapharm, Saarbruecken, Germany) were applied for protection. Two weeks after injection the fundus autofluorescence was measured in vivo. Afterwards the mice were killed and the eyes from twelve mice were enucleated and prepared for histology.

### Sample Preparation for Fluorescence and Electron Microscopy

Mouse eyes were fixed, embedded in epon resin and sectioned according to standard procedures. For fluorescence analysis, post-fixation and staining with heavy metals was omitted. Semi-thin sections (500 nm) were prepared and cover-slipped with Dako fluorescent mounting medium. For electron microscopy, ultra-thin sections (70 nm) were collected on formvar-coated slot grids stained with lead citrate and investigated on a Zeiss 900 electron microscope (Zeiss, Jena, Germany).

### Fluorescence Microscopy

Specimens were investigated with a Zeiss Axioplan 2 microscope (Zeiss, Jena, Germany) equipped with a Lumencor Sola SE II NIR (Beaverton, OR, USA) light source and using a x63 objective. Filter sets were a custom made lipofuscin filter set (excitation 370/36 nm, emission 575/15, 400 nm beam splitter) for SW-AF and a commercial Cy7 filter set (excitation 708/75 nm, emission 809/81 nm, 757 nm beam splitter) for NIR-AF, respectively. The lipofuscin filter set is designed to fit the reported excitation and emission maximums for lipofuscin. Binning x2 was applied for all images and acquisition times, as well as microscope and software settings were held constant for any set of samples to allow comparison of fluorescence intensities.

Correlative fluorescence and electron microscopy

To accommodate the requirements of both fluorescence and electron microscopy, ultra-thin sections with a thickness of 150 nm were used. This allows improved detection of fluorophores compared to standard 70 nm ultra-thin sections (as routinely used for electron microscopy), while still being thin enough to allow electron microscopic investigation (as opposed to 500 nm semi-thin sections that would be too thick to be penetrated by the electron beam). Non-osmicated sections were collected on formvar-coated mesh grids. Grids were placed on glass slides in a drop of water, coverslipped, and investigated with light and fluorescence microscopy as described before. Acquisition times had to be prolonged compared to 500 nm thick sections and additionally the auto-contrast function of the camera capture software was used. Signal-to-noise ratio was rather low due to the limited section thickness, so five pictures per channel were averaged using Photoshop CS2. Since sections tend to adhere to the coverslips, grids had to be carefully manipulated off the coverslips with forceps to not destroy the sections. Nevertheless, sections are stressed by being coverslipped and easily damaged, therefore post-staining with uranyl acetate and lead citrate was ultimately omitted, since this often resulted in complete loss of the sections. Grids were air dried and investigated by electron microscopy as described before. The lack of any heavy-metal staining results in low contrast, but melanosomes and lipofuscin granules are still reliably identifiable.

### Fundus Autofluorescence Image Acquisition

Engineering, Heidelberg, Germany) was used for fundus AF image acquisition as reported previously (Charbel Issa et al., 2013). Twelve Abca4^{(-/-)} mice were anesthetized using the three-component narcosis as described earlier. Methocel was applied on the surface of the cornea after full dilation of pupils (diameters >2 mm). To apply the Spectralis to the analysis of mouse retina, we fixed a 78-dpt noncontact slit lamp lens (Volk Optical, Inc., Mentor, OH 44060, USA) directly in front of the device. Additionally, a custom-made contact lens (100 dpt) was positioned onto the cornea. The mouse was placed on a three-dimensional platform that is adjustable to acquire clear scanning images. The near-infrared reflectance (NIR-R) mode was performed first to align the camera and acquire well-focused images centered on the optic nerve head (ONH). Short-wavelength autofluorescence (SW-AF) images and near-infrared autofluorescence images (NIR-AF) were recorded simultaneously with excitation of the 488-nm laser and 788-nm laser, respectively. All the fundus fluorescence images were recorded with 55 and 30° angles of view, a 768×768 pixel image size, and a detector sensitivity setting at 100 after the fundus was exposed to the blue laser for 20 seconds. The automatic real-time function was activated for image capture. Fifteen consecutive frames were captured in the video format and then the averaged images were also saved in the “non-normalization” mode for the quantitation of fundus AF. Normalized images as the average of 100 successive frames were also captured. For correlative analysis, the normalized images with different excitations were coded with different colors and merged using Image J software.

### Results

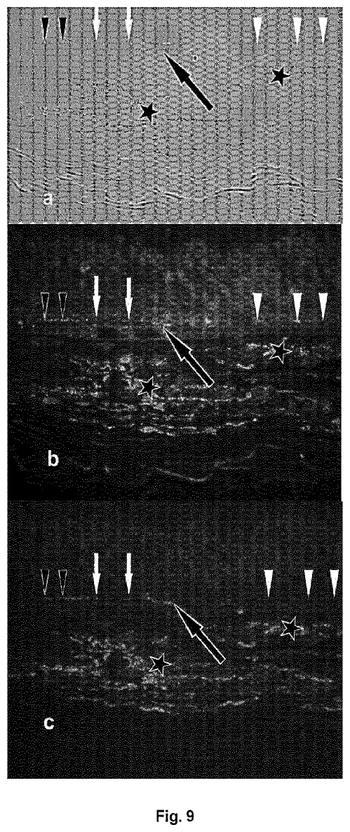

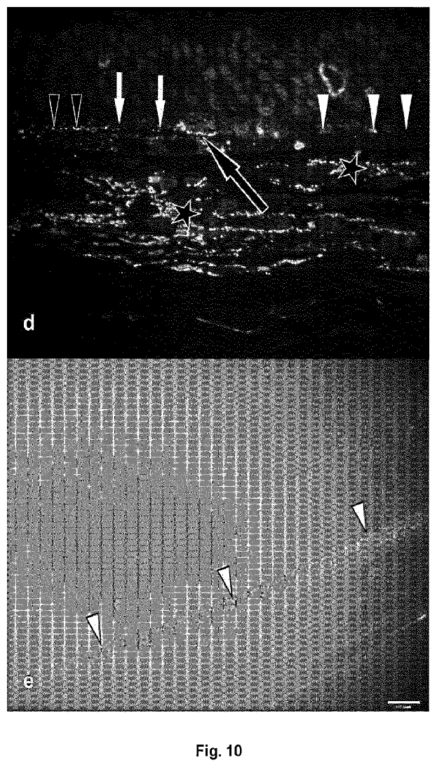

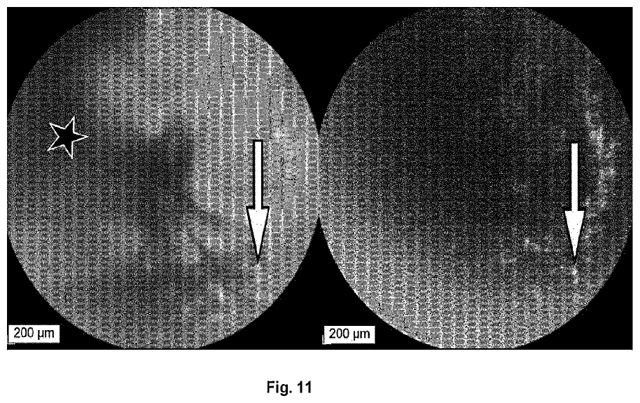

The results are shown in FIGS. 9 to 11. In summary, it is demonstrated that tyrosinase vector induces melanogenesis in the retinal pigment epithelium of albino Abca4^{(-/-)} mice. The melanin granules are involved in chemical reactions with the lipofuscin residues that are present in high concentration in the RPE of these mice. That the melanosomes have underwent a chemical reaction is shown by the induction of their autofluorescence in the NIR range. Oxidation of melanin causes NIR-AF (see e.g. Taubitz, T., et al., Age, lipofuscin and melanin oxidation affect fundus near-infrared autofluorescence. EBioMedicine, 2019. 48: p. 592-604 and Taubitz, T., et al., Ultrastructural alterations in the retinal pigment epithelium and photoreceptors of a Stargardt patient and three Stargardt mouse models: indication for the central role of RPE melanin in oxidative stress. PeerJ, 2018. 6: p. e5215).

Finally the substitution with melanin removed lipofuscin from the RPE of these mice, thus demonstrating that melanin is capable of removing lipofuscin and this represents a promising approach for treating lipofuscin-associated diseases.

### Example 5—Different Ultrastructural Findings in the RPE of Albino and Pigmented Abca4^{-/-} Mice without Treatment

### Electron Microscopic Investigation and Quantification of Vacuoles Containing TLMs

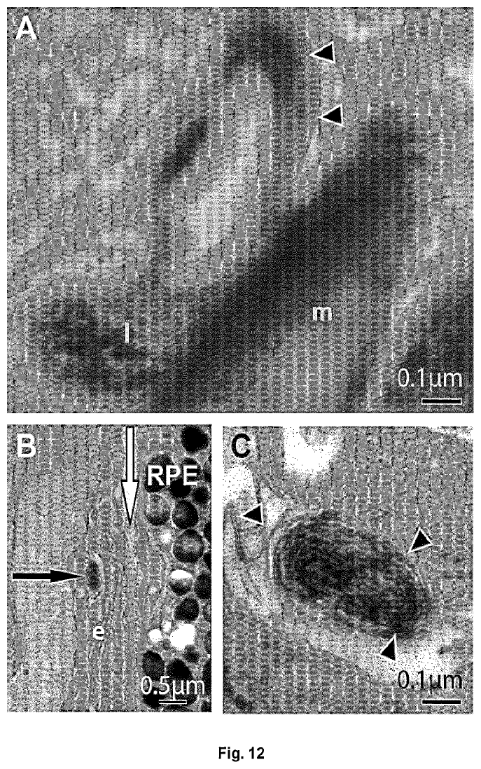

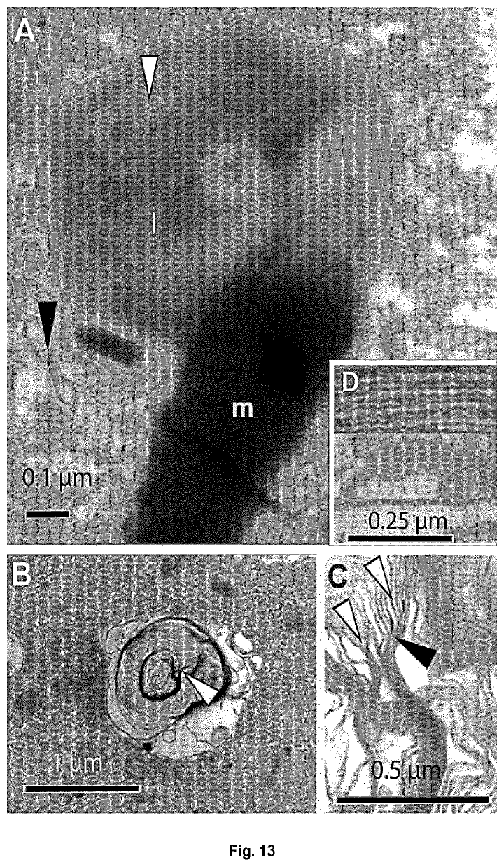

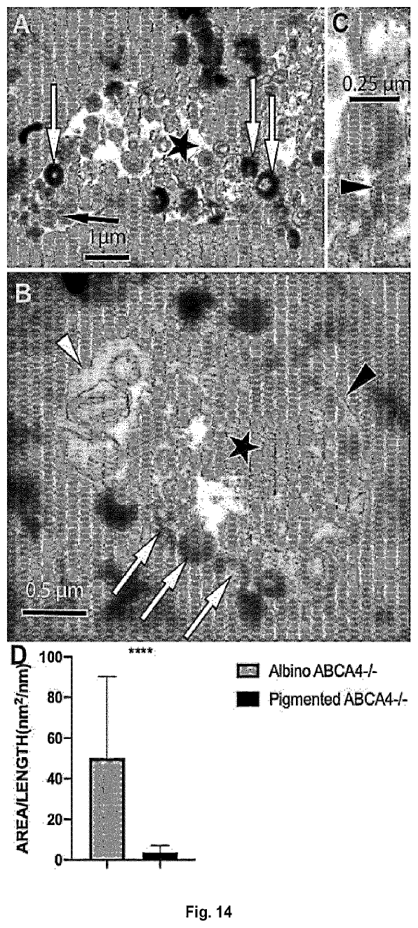

Sections were examined comprehensively for changes in RPE and choroid. The area of vacuole-like structures (described by Taubitz, T., et al., Ultrastructural alterations in the retinal pigment epithelium and photoreceptors of a Stargardt patient and three Stargardt mouse models: indication for the central role of RPE melanin in oxidative stress. PeerJ, 2018. 6: p. e5215) and the length of the RPE layer were measured in 7 months old pigmented and albino Abca4/-mice. Thin lamellar membranes were measured in electron micrographs at 140000 fold magnification. All measurements were performed with imageSP Software (Minsk, Belarus). The results are depicted in FIGS. 12 to 14.

In summary, these results show that accumulation of TLMs in RPE cells is much more frequent in the albinos compared to the pigmented mice showing a role of melanin in the removal of TLMs. The therapeutic use of melanin isalso supported by removal of TLMs. Further, TLMs can thus will be diagnostic for lipofuscin-associated diseases.

### Example 6—the Catalytic Effects of Melanin in Combination with a Superoxide- and/or a NO-Generator in the Promotion of Oxidation of A2E in Solution

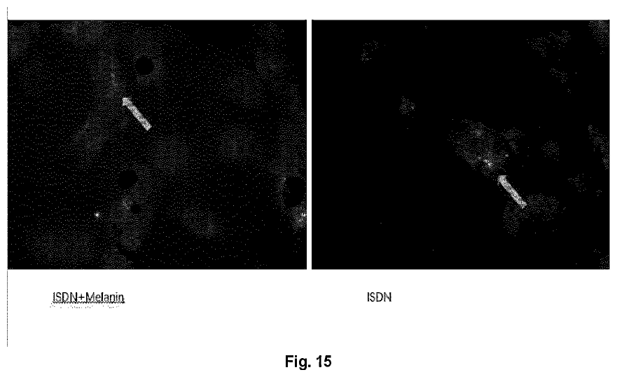

To show the effect of an NO-generator in combination with melainin, the nitric oxide donor isosorbid dinitrate (ISDN, Ratiopharm GmbH, Ulm, Germany) was combined with melanin and incubated with A2E as shown in FIG. 15. FIG. 15 shows a surprisingly strong fluorescent signal in the ISDN+Melanin treatment group than in the ISDN treatment group alone.

To show the effect of a superoxide generator in combination with melanin, melanin combined with the superoxide generator soraprazan and incubated with A2E and compared with melanin alone, and with soraprazan alone.

For this, the solutions or suspensions were prepared in acetonitrile. Solutions were prepared in 10× stock solutions and diluted in the incubation vial to the final concentrations. Incubations in light were made in clear glass HPLC sample vials (VWR) at room temperature under normal room lighting (300 lux).

Samples were taken and analysed directly for A2E using LCMSMS. A2E was quantified based on a standard curve using authentic A2E solutions in acetonitrile vs. peak area in the same LCMSMS system.

Melanin was provided at a concentration of 0.1 mg/mL. Soraprazan was provided at 1 μM. The combinations were made in the same way (0.1 mg/mL melanin/1 μM soraprazan). Below, the degradation data in acetonitrile for A2E vs. time are shown. A2E always starts at 10 μM Samples were taken at Oh and 2h.

| | | Time after mixing (h) (0, 2 h) | |
|---|---|---|---|
| | | A2E μM | A2E μM |
| | Concentration | 0 h | 2 h |
| a. soraprazan - μM | 1 | 10 | 8.29 |
| b. melanin - mg/mL | 0.1 | 10 | 8.49 |
| c. soraprazan/melanin | a + b | 10 | 6.28 |

The data show a synergistic effect of the combination of soraprazan with melanin.

1. A composition for use in the treatment of a lipofuscin-associated disease, wherein the composition comprises melanin and/or induces melanogenesis.

2. The composition for use according to claim 1, wherein melanin interacts with lipofuscin.

3. The composition for use according to claim 1 or 2, wherein melanin degrades and/or removes lipofuscin.

4. The composition for use according to any one of claims 1 to 3, wherein thin lamellar membranes (TLMs) are removed.

5. The composition for use according to any one of claims 1 to 4, wherein the lipofuscin associated disease is a disease of the eye or the CNS.

6. The composition for use according to claim 5, wherein the lipofuscin associated disease is a disease of the eye and is selected from age-related macular degeneration, Stargardt's disease, Best's disease or Retinitis pigmentosa.

7. The composition for use according to claim 6, wherein the age-related macular degeneration is dry AMD or wet AMD, preferably dry AMD.

8. The composition for use according to any one of claims 1 to 7, wherein the treatment comprises administering an injection of the composition to a patient.

9. A composition for use in the treatment of a lipofuscin-associated disease, wherein the composition comprises a combination of melanin and/or a melanin inducing agent with a superoxide-generator and/or an NO-generator.

10. A composition for use in the treatment of a lipofuscin-associated disease, wherein the composition comprises a combination of melanin and/or a melanin inducing agent with soraprazan and/or ISDN.
